(19) United States
(12) Patent Application Publication
(10) Pub. No.: US 2020/0215013 A1
(43) Pub. Date: Jul. 9, 2020

(54) COMBINATION COMPRISING DECANOIC ACID FOR THE TREATMENT OF EPILEPSY

(71) Applicants: VITAFLO INTERNATIONAL LTD, Liverpool Merseyside (GB); UCL BUSINESS LTD., London (GB)

(72) Inventors: Robin Simon Brooke Williams, Egham (GB); Matthew Walker, London (GB)

(21) Appl. No.: 16/624,381

(22) PCT Filed: Jun. 28, 2018
(86) PCT No.: PCT/EP2018/067355

§ 371 Date: Dec. 19, 2019

(30) Foreign Application Priority Data
Jun. 29, 2017 (EP) 17178751.8

(51) Int. Cl. A61K 31/20; A61K 31/4412; A23L 33/12; A61K 9/107; A61K 9/14

(52) U.S. Cl. CPC A61K 31/20; A61K 31/4412; A61K 9/14; A61K 9/107; A23L 33/12

(57) ABSTRACT

Decanoic acid for use in treating epilepsy wherein the decanoic acid is used in combination with perampanel, or a pharmaceutically acceptable salt thereof, or wherein the decanoic acid is used in combination with an AMPA receptor inhibitor that binds to the same AMPA receptor site as perampanel.

### FIELD OF THE INVENTION

The present invention generally relates to a combination of (i) decanoic acid and perampanel, or a pharmaceutically acceptable salt thereof or (ii) decanoic acid and an AMPA receptor inhibitor that binds to the same AMPA receptor site as perampanel. In particular, the present invention provides said combinations for treating epilepsy.

### BACKGROUND TO THE INVENTION

Epilepsy covers a broad range of neurological disorders that are characterised by seizures. Seizures result from abnormal neuronal activity and manifest in a number of ways, including convulsions and loss of awareness. In many cases epilepsy can be managed by the use of anti-convulsive medication. However for a proportion of patients with epilepsy, treatment with conventional drugs can have minimal effect upon seizure activity. Although surgery is an option for treating patients suffering from certain seizures, for many individuals successful management can be achieved less invasively with the ketogenic diet.

The medium chain triglyceride (MCT) ketogenic diet was first identified as a treatment for refractory epilepsy in 1971. It has provided one of the most effective therapeutic approaches for children with drug resistant epilepsy (Liu, Epilepsia 2008; 49 Suppl. 8: 33-36) and has been demonstrated to be effective in childhood epilepsy in a randomised control trial (Neal et al., Epilepsia 2009; 50: 1109-1117). However, the diet has adverse gastro-intestinal related side effects, such as diarrhea, vomiting, bloating, and cramps (Liu, Epilepsia 2008; 49 Suppl 8: 33-36.). Furthermore, it has also been shown that there is a high attrition rate for the diet, due to many patients finding the diet difficult to tolerate (Levy et al., Cochrane Database Syst Rev 2012; 3: CD001903).

Although ketone bodies resulting from the ketogenic diet have been postulated to play a therapeutic role, seizure control is poorly correlated with ketone body levels (Likhodii et al., Epilepsia 2000; 41: 1400-1410; Thavendiranathan et al., Exp Neurol 2000; 161: 696-703). In addition to ketones, the diet also causes an increase in plasma levels of the two fatty acids provided in MCT oil, the straight chain, ten carbon decanoic acid, and the eight carbon octanoic acid (Haidukewych et al., Clin Chem 1982; 28: 642-645). Recently, it has been established that decanoic acid, but not octanoic acid, has anti-seizure effects at clinically relevant concentrations in vitro and in vivo (Chang et al., Neuropharmacology 2013; 69: 105-114; Wlaz et al., Progress in Neuropsychopharmacology & Biological Psychiatry 2014). α-amino-3-hydroxy-5-methyl-4-isoxazolepropionic acid receptors (AMPA receptors) play a key role in generating and propagating epileptic activity and in the long-term, adaptive cellular plasticity associated with epileptogenesis (Chapman, J Nutr 2000; 130: 1043S-1045S; Rogawski and Donevan, Adv Neurol 1999; 79: 947-963). The receptors are present in all areas relevant to epilepsy, including the cerebral cortex, amygdala, thalamus and hippocampus. Furthermore, AMPA receptor antagonists have a broad spectrum of anticonvulsant activity in various in vitro and in vivo epilepsy models (Rogawski., Epilepsy Curr 2011; 11: 56-63).

We have recently demonstrated that decanoic acid inhibits AMPA receptors (Chang et al., Brain. 2016 February; 139(2): 431-443).

Perampanel (Fycompa) is a non-competitive AMPA receptor antagonist which has been licensed as an adjunctive treatment for partial-onset and primary generalised tonic-clonic seizures (Frampton J E. 2015. Drugs 75: 1657-68). Adjunctive perampanel has also been found to be effective in children with refractory partial-onset seizures and for tonic-clonic seizures in idiopathic generalized epilepsy (Heyman E. Developmental Medicine & Child Neurology 2017, 59: 441-444). However, it has dose-dependent behavioural side-effects, limiting its use in some patients (Rugg-Gunn F. 2014. Epilepsia 55 Suppl 1: 13-5).

The most common side-effects reported in patients receiving perampanel have been dizziness, somnolence, fatigue, irritability, nausea and falls, but of particular concern to patients were the cognitive and psychiatric side effects of the drug. Rugg-Gunn F. describes how overall, depression and aggression were reported more frequently in patients taking perampanel, particularly at higher doses, than in patients taking placebo. Heyman E. reports that perampanel is associated with a relatively high rate of behavioural adverse effects mostly in adolescents with refractory epilepsy.

There remains a need for improved medicaments to treat epilepsy.

### SUMMARY OF THE INVENTION

The present inventors have surprisingly shown a synergistic interaction between perampanel and decanoic acid in direct AMPA receptor inhibition and in seizure control. These findings provide a role for combination treatment using perampanel or an AMPA receptor inhibitor that binds to the same AMPA receptor site as perampanel and decanoic acid.

### STATEMENTS OF THE INVENTION

According to a first aspect of the invention there is provided decanoic acid for use in treating epilepsy wherein the decanoic acid is used in combination with perampanel, or a pharmaceutically acceptable salt thereof, or wherein the decanoic acid is used in combination with an AMPA receptor inhibitor that binds to the same AMPA receptor site as perampanel.

According to another aspect of the invention there is provided perampanel or a pharmaceutically acceptable salt thereof for use in treating epilepsy wherein the perampanel (or the pharmaceutically acceptable salt thereof) is used in combination with decanoic acid.

According to another aspect of the invention there is provided an AMPA receptor inhibitor that binds to the same AMPA receptor site as perampanel for use in treating epilepsy, wherein the AMPA receptor inhibitor is used in combination with decanoic acid.

The decanoic acid and perampanel, or a pharmaceutically acceptable salt thereof, may be administered simultaneously, separately or sequentially, or the decanoic acid and the AMPA receptor inhibitor may be administered simultaneously, separately or sequentially. If the agents are not administered simultaneously, they are administered within a time interval that allows the agents to show a synergistic effect.

According to another aspect of the invention there is provided a combination of (i) decanoic acid and perampanel, or a pharmaceutically acceptable salt thereof, or (ii) decanoic acid and an AMPA receptor inhibitor that binds to the same AMPA receptor site as perampanel for use in treating epilepsy.

According to another aspect of the invention there is provided a product comprising (i) decanoic acid and perampanel, or a pharmaceutically acceptable salt thereof, or (ii) decanoic acid and an AMPA receptor inhibitor that binds to the same AMPA receptor site as perampanel; as a combined preparation for simultaneous, separate or sequential use in treating epilepsy.

According to another aspect of the invention there is provided a composition comprising (i) decanoic acid and perampanel, or a pharmaceutically acceptable salt thereof, or (ii) decanoic acid and an AMPA receptor inhibitor that binds to the same AMPA receptor site as perampanel. In one embodiment the composition is for use in treating epilepsy.

According to another aspect of the invention there is provided a kit comprising (i) decanoic acid and perampanel, or a pharmaceutically acceptable salt thereof, or (ii) decanoic acid and an AMPA receptor inhibitor that binds to the same AMPA receptor site as perampanel. The kit may optionally include instructions for the simultaneous, sequential or separate administration of (i) the decanoic acid and perampanel, or a pharmaceutically acceptable salt thereof, or (ii) the decanoic acid and the AMPA receptor inhibitor, to a patient in need thereof.

According to another aspect of the invention there is provided a method for treating epilepsy which comprises the step of administering decanoic acid to a patient in need thereof, wherein the decanoic acid is administered to the patient in combination with perampanel, or a pharmaceutically acceptable salt thereof, or wherein the decanoic acid is administered to the patient in combination with an AMPA receptor inhibitor that binds to the same AMPA receptor site as perampanel.

According to another aspect of the invention there is provided a method for treating epilepsy which comprises the step of administering perampanel or a pharmaceutically acceptable salt thereof to a patient in need thereof, wherein the perampanel, or a pharmaceutically acceptable salt thereof, is administered to the patient in combination with decanoic acid.

According to another aspect of the invention there is provided a method for treating epilepsy which comprises the step of administering an AMPA receptor inhibitor that binds to the same AMPA receptor site as perampanel to a patient in need thereof, wherein the AMPA receptor inhibitor is administered to the patient in combination with decanoic acid.

According to another aspect of the invention there is provided a method for treating epilepsy which comprises the step of administering a composition comprising (i) perampanel, or a pharmaceutically acceptable salt thereof, and decanoic acid or (ii) an AMPA receptor inhibitor that binds to the same AMPA receptor site as perampanel and decanoic acid to a patient in need thereof.

According to another aspect of the present invention there is provided a combination of (i) decanoic acid and perampanel, or a pharmaceutically acceptable salt thereof, or (ii) decanoic acid and an AMPA receptor inhibitor that binds to the same AMPA receptor site as perampanel, for use in inhibiting AMPA receptors in a subject in need of said inhibition. Said subject may be suffering from epilepsy. According to another aspect of the present invention said subject may be suffering from ischemia, amyotrophic lateral sclerosis (ALS), cancer or Alzheimer's Disease.

In one embodiment, the subject to be treated in the present invention has been identified as a subject that would respond to AMPA receptor inhibition.

According to another aspect of the invention there is provided decanoic acid for use in treating ischemia, amyotrophic lateral sclerosis (ALS), cancer or Alzheimer's Disease wherein the decanoic acid is used in combination with perampanel or a pharmaceutically acceptable salt thereof, or wherein the decanoic acid is used in combination with an AMPA receptor inhibitor that binds to the same AMPA receptor site as perampanel.

According to another aspect of the invention there is provided perampanel or a pharmaceutically acceptable salt thereof for use in treating ischemia, amyotrophic lateral sclerosis (ALS), cancer or Alzheimer's Disease wherein the perampanel, or the pharmaceutically acceptable salt thereof, is used in combination with decanoic acid.

According to another aspect of the invention there is provided an AMPA receptor inhibitor that binds to the same AMPA receptor site as perampanel for use in treating ischemia, amyotrophic lateral sclerosis (ALS), cancer or Alzheimer's Disease wherein the AMPA receptor inhibitor is used in combination with decanoic acid.

According to another aspect of the invention there is provided a method for treating ischemia, amyotrophic lateral sclerosis (ALS), cancer or Alzheimer's Disease which comprises the step of administering decanoic acid to a patient in need thereof, wherein the decanoic acid is administered to the patient in combination with perampanel or a pharmaceutically acceptable salt thereof, or wherein the decanoic acid is administered in combination with an AMPA receptor inhibitor that binds to the same AMPA receptor site as perampanel.

According to another aspect of the invention there is provided a method for treating ischemia, amyotrophic lateral sclerosis (ALS), cancer or Alzheimer's Disease which comprises the step of administering perampanel or a pharmaceutically acceptable salt thereof to a patient in need thereof, wherein the perampanel or a pharmaceutically acceptable salt thereof is administered to the patient in combination with decanoic acid.

According to another aspect of the invention there is provided a method for treating ischemia, amyotrophic lateral sclerosis (ALS), cancer or Alzheimer's Disease which comprises the step of administering an AMPA receptor inhibitor that binds to the same AMPA receptor site as perampanel, to a patient in need thereof, wherein the AMPA receptor inhibitor is administered to the patient in combination with decanoic acid.

According to another aspect of the invention there is provided a method for treating ischemia, amyotrophic lateral sclerosis (ALS), cancer or Alzheimer's Disease which comprises the step of administering a composition comprising (i) perampanel or a pharmaceutically acceptable salt thereof and decanoic acid or (ii) an AMPA receptor inhibitor that binds to the same AMPA receptor site as perampanel and decanoic acid to a patient in need thereof.

The treatment of epilepsy referred to herein may comprise controlling epileptic seizures.

The decanoic acid referred to herein may be in the form of a triglyceride.

Alternatively the decanoic acid referred to herein may be in the form of a pharmaceutically acceptable salt or an ester. Salts and esters of decanoic acid are also known in the art as decanoates or caprates.

The decanoic acid may be comprised in a composition, for example a pharmaceutical composition. As discussed below in more detail, the perampanel or a pharmaceutically acceptable salt thereof may be present in the same composition as decanoic acid or a different composition. Alternatively, the AMPA receptor inhibitor that binds to the same AMPA receptor site as perampanel may be present in the same composition as decanoic acid or a different composition.

In one embodiment, the decanoic acid makes up at least 50, 60, 70, 80, 85, 90, 95 or 99%, or 100% by weight of the total fatty acid content of the composition. In one embodiment, the decanoic acid is in the form of medium chain triglycerides wherein said triglycerides make up at least 50, 60, 70, 80, 85, 90, 95 or 99%, or 100% of the total fat content of the composition. In one embodiment, substantially all the fatty acid moieties of the MCTs are octanoic acid and decanoic acid moieties. In one embodiment, substantially all the fatty acid moieties of the MCTs are decanoic acid moieties.

In one embodiment, the composition is substantially free of mono- or poly-unsaturated fatty acids. In one embodiment, the composition is in the form of an oil-in-water emulsion, a powder or a food stuff. In one embodiment, the decanoic acid is present in the composition at 5 g/l to 500 g/l, 5 g/l to 200 g/l, 5 g/l to 100 g/l, 5 g/l to 50 g/l, 5 g/l to 30 g/l, 5 g/l to 20 g/l, 10 g/l to 500 g/l, 10 g/l to 200 g/l, 10 g/l to 100 g/l, 10 g/l to 50 g/l, 10 g/l to 30 g/l or 10 g/l to 20 g/l.

For example, decanoic acid may be present in a composition at about 5 g/l, about 10 g/l, about 15 g/l, about 20 g/l, about 30 g/l, about 40 g/l, about 50 g/l, about 60 g/l, about 70 g/l, about 80 g/l, about 90 g/l, about 100 g/l, about 110 g/l, about 120 g/l, about 130 g/l, about 140 g/l, about 150 g/l, about 175 g/l, about 200 g/l, about 225 g/l, about 250 g/l or about 500 g/l.

In another embodiment, the decanoic acid is present in a composition which is free, or substantially free, of carbohydrate and protein, e.g. the composition has less than 2%, 0.5% or 0.1% carbohydrate and protein by weight. In one embodiment, the weight amounts of lipid to the sum of proteins and carbohydrates in the composition is 1-5 to 1. For example, the weight amounts of lipid to the sum of proteins and carbohydrates may be 1 to 1, 2 to 1, 3 to 1, 4 to 1, 5 to 1, 2.4-4.0 to 1, or 2.6-3.8 to 1.

The decanoic acid may be comprised within an oil-in-water emulsion. In one embodiment, the emulsion comprises decanoic acid in the form of medium chain triglycerides wherein said medium chain triglycerides make up at least 50, 60, 70, 80, 85, 90, 95 or 99%, or 100% of the total fat content of the composition. In one embodiment, all, or substantially all, of the fatty acid moieties of the MCTs are decanoic acid moieties and octanoic acid moieties. In one embodiment, all, or substantially all, of the fatty acid moieties of the MCTs are decanoic acid moieties. The emulsion may comprise substantially no protein or carbohydrate. In one embodiment, the total fat content of the oil in water emulsion is 5 to 40 g/100 ml, for example 5 to 30 g/100 ml, 5 to 25 g/100 ml, 10 to 25 g/100 ml or 10 to 20 g/100 ml or 15 to 25 g/100 ml. In one embodiment, the energy value of the emulsion is between 50 to 300 kcal per 100 ml, for example, 100 to 300 kcal per 100 ml, 50 to 200 kcal per 100 ml, 150 to 250 kcal per 100 ml or 170 to 200 kcal per 100 ml.

In one embodiment, the decanoic acid is present in a composition which is in powdered form.

In another embodiment, the decanoic acid is present in a composition which is in a spray dried form.

In another embodiment, the decanoic acid is comprised within a fortifying food or drink.

In another embodiment, the decanoic acid is present within a food stuff.

In another embodiment, the decanoic acid is present within a medical food.

In another embodiment, the decanoic acid is present within a tube feed.

In another embodiment, the decanoic acid is comprised within a beverage, mayonnaise, salad dressing, margarine, low fat spread, dairy product, cheese spread, processed cheese, dairy dessert, flavoured milk, cream, fermented milk product, cheese, butter, condensed milk product, ice cream mix, soya product, pasteurised liquid egg, bakery product, confectionary product, confectionary bar, chocolate bar, high fat bar, UHT pudding, pasteurised pudding, gel, jelly, yoghurt, or a food with a fat-based or water-containing filling.

In another embodiment, the decanoic acid is comprised within a pharmaceutical composition. The pharmaceutical composition may comprise one or more suitable pharmaceutically acceptable carriers, diluents and/or excipients.

### DESCRIPTION OF THE DRAWINGS

FIG. 1. AMPA (GluA2/3, GluA1/2, or GluA3) receptors were expressed in Xenopus oocytes, and perfused with L-glutamate (100 μM) and the indicated compound, unless stated otherwise. Currents were recorded using TEVC. (A) Representative current traces of inhibitory dose-response curves for perampanel on GluA1/2 and GluA2/3 receptors. (B) Dose-response curve showing inhibition of GluA1/2 (N=10) and GluA2/3 (N=6) by perampanel, with respective IC₅₀ values shown in bar graph inset. (C) Effect of varying perampanel concentrations on glutamate EC₅₀ against GluA1/2. Points were normalised to maximal response and represent the mean and SEM of 6 (glutamate only) and 5 (with 2.5 μM and 5 μM perampanel). (D) Quantitative representation of mean currents recorded in wildtype and mutant GluA3 normalised to maximal responses in the presence of perampanel (N=5 for wildtype and N=6 for mutant) and decanoic acid, DA (N=4 for both wildtype and mutant).

FIG. 2. AMPA (GluA2/3, GluA1/2, or GluA3) receptors were expressed in Xenopus oocytes, and perfused with L-glutamate (100 μM) and the indicated compound, unless stated otherwise. Currents were recorded using TEVC. (A) Representative current traces of inhibitory dose-response curves for decanoic acid (DA) on GluA2/3 receptors at 1 μM or 4 μM perampanel. Dose-response inhibition curves for decanoic acid at 1 μM or 4 μM perampanel on (B) GluA2/3 and (C) GluA1/2 receptors. Points were normalised to maximal response to L-glutamate and solvent, 1 μM perampanel or 4 μM perampanel and represent means and SEM of 8 to 13 readings. Inserts show respective IC₅₀ values in the presence of perampanel. (D) Representative current traces of inhibitory dose-response curves for perampanel on GluA2/3 receptors at 50 μM to 100 μM decanoic acid. (E),(F) Dose-response inhibition curves for perampanel at 50 μM and 100 μM DA on GluA2/3 (E) and GluA1/2 (F) receptors. Points were normalised to maximal response to L-glutamate with solvent, 50 μM and 100 μM DA and represent means and SEM of 8 to 13 readings. Inserts show respective perampanel IC₅₀ values in the presence of DA. Scale bars correspond to 150 nA in GluA1/2 and 30 nA in GluA2/3 (A), 50 nA (E) and 200 nA (H). Scale bars correspond to 60 nA for +1 μM perampanel and 75 nA for +4 μM perampanel (K) and 200 nA (L).

FIG. 3. (A) Epileptiform (paroxysmal) activity was induced in rat entorhinal cortex-hippocampal slices by application of PTZ (2 mM) and [K+] (to 6 mM), and recorded over time at fixed perampanel and increasing decanoic acid (DA) concentration. (B) Epileptiform activity was normalised to activity in absence or presence of each concentration (100 nM and 500 nM) and shown at variable DA concentrations. Insert shows DA IC₅₀ data for epileptiform activity with perampanel. Data is derived from at least three biological repeats.

### DETAILED DESCRIPTION

### Combination

According to the present invention, decanoic acid is used in combination with perampanel, or a pharmaceutically acceptable salt thereof, or decanoic acid is used in combination with an AMPA receptor inhibitor that binds to the same AMPA receptor site as perampanel.

As used herein, the term “combination” or phrases “in combination”, “used in combination with” or “combined preparation” refer to the combined administration of (i) decanoic acid and perampanel, or a pharmaceutically acceptable salt thereof, or (ii) decanoic acid and an AMPA receptor inhibitor that binds to the same AMPA receptor site as perampanel, wherein the decanoic acid and the perampanel, or a pharmaceutically acceptable salt thereof may be administered simultaneously, sequentially or separately, or wherein the decanoic acid and the AMPA receptor inhibitor may be administered simultaneously, sequentially or separately.

As used herein, the term “simultaneous” or “simultaneously” is used to mean that the agents are administered concurrently, i.e. at the same time.

The term “sequential” or “sequentially” is used to mean that the two agents are administered one after the other, where either the decanoic acid is administered first or the perampanel, a pharmaceutically acceptable salt thereof, or the AMPA receptor inhibitor is administered first.

The term “separate” or “separately” is used to mean that the two agents are administered independently of each other but within a time interval that allow the agents to show a synergistic effect. Thus, administration “separately” may permit one agent to be administered, for example, within 1 minute, 5 minutes or 10 minutes after the other, provided that the agents show a synergistic effect.

The agents may be administered either as separate formulations or as a single combined formulation. When combined in the same formulation, it will be appreciated that the two agents must be stable and compatible with each other and any other components of the formulation.

When the agents are co-formulated, i.e. in the same composition or formulation, they can only be administered simultaneously. When the agents are formulated in separate compositions or formulations, they can be administered simultaneously, sequentially or separately.

Simultaneous administration of the agents in the same formulation or in separate formulations can also be described as the co- or joint administration of the two agents.

In one embodiment, decanoic acid and perampanel, or a pharmaceutically acceptable salt thereof, are in admixture. In another embodiment, the decanoic acid and perampanel, or pharmaceutically acceptable salt thereof, are present in the form of a kit comprising a preparation of decanoic acid and perampanel, or a pharmaceutically acceptable salt thereof, and, optionally, instructions for the simultaneous, sequential or separate administration of the preparations to a patient in need thereof.

In an alternative embodiment, decanoic acid and an AMPA receptor inhibitor that binds to the same AMPA receptor site as perampanel, are in admixture. In another embodiment, the decanoic acid and the AMPA receptor inhibitor that binds to the same AMPA receptor site as perampanel, are present in the form of a kit comprising a preparation of decanoic acid and the AMPA receptor inhibitor, and, optionally, instructions for the simultaneous, sequential or separate administration of the preparations to a patient in need thereof.

In another embodiment the decanoic acid and perampanel, or pharmaceutically acceptable salt thereof are present in a product as a combined preparation for simultaneous, separate or sequential use in treating epilepsy or in inhibiting AMPA receptors in a subject in need of said inhibition.

Alternatively, the decanoic acid and the AMPA receptor inhibitor are present in a product as a combined preparation for simultaneous, separate or sequential use in treating epilepsy or in inhibiting AMPA receptors in a subject in need of said inhibition.

### Decanoic Acid and Compositions Comprising the Same

Decanoic acid (also known as capric acid) is a saturated fatty acid of the formula CH₃(CH₂)COOH.

It will be appreciated that the decanoic acid may be in free form (or a salt thereof) or in the form of, for example, triglycerides, diacyl-glycerides, monoacyl-glycerides, with triglycerides being generally preferred.

A medium-chain triglyceride (MCT) is a triglyceride in which all three fatty acid moieties are medium-chain fatty acid moieties, medium-chain fatty acids (MCFA) are fatty acids that have 6 to 12 carbon atoms, although fatty acids with 8 and 10 carbon atoms (i.e. octanoic acid and decanoic acid) are preferred and may be referred to herein as C8 fatty acids or C8, and C10 fatty acids or C10.

The term “fatty acid moiety” refers to the part of the MCT that originates from a fatty acid in an esterification reaction with glycerol. For example, an esterification reaction between glycerol and only decanoic acid would result in a MCT with decanoic acid moieties.

Homotriglycerides (i.e. all of the fatty acid moieties of the MCT are of the same identity, for example a C10 homotriglyceride may comprise 3 decanoic acid moieties) and/or heterotriglycerides (i.e. the fatty acid moieties of the MCT are not all the same identity) may be used in the present invention. Preferred heterotriglycerides are heterotriglycerides made up of octanoic acid and decanoic acid moieties.

The decanoic acid (or triglycerides comprising decanoic acid) may be in the form of a composition. The perampanel may be in the same composition or administered separately.

In one embodiment, the composition is free from or substantially free from fatty acid moieties that are not decanoic acid or octanoic acid. In one embodiment, the composition is free from or substantially free from fatty acid moieties that are not decanoic acid. In one embodiment, the composition is free from or substantially free from MCTs comprising fatty acid moieties that are not decanoic acid and octanoic acid. In one embodiment, the composition is free from or substantially free from MCTs comprising fatty acid moieties that are not decanoic acid. However, there may be traces of such MCTs (e.g., less than 3, 2, 1 or 0.5 wt %).

Examples of natural sources of MCT include plant sources such as coconuts, coconut oil, palm kernels, palm kernel oils, and animal sources such as milk. Decanoic acid forms about 5-8% of the fatty acid composition of coconut oil.

MCTs may also be synthesised by esterification of glycerol with one or more medium-chain fatty acids (MCFA). For example, MCT-C10 can be synthesised by esterification of glycerol with decanoic acid.

The composition comprising decanoic acid may also comprise long chain triglycerides (LCTs). Preferably the LCTs are at a level of less tan 5%, 2%, 1%, 0.5% or 0.1 wt % of the composition. In one embodiment, no LCTs are present in the composition.

The composition may further comprise substances such as minerals, vitamins, salts, functional additives including, for example, palatants, colorants, emulsifiers, antimicrobial or other preservatives. Minerals that may be useful in such compositions include, for example, calcium, phosphorous, potassium, sodium, iron, chloride, boron, copper, zinc, magnesium, manganese, iodine, selenium, chromium, molybdenum, fluoride and the like. Examples of vitamins that may be useful in compositions described herein include water soluble vitamins (such as thiamin (vitamin B1), riboflavin (vitamin B2), niacin (vitamin B3), pantothenic acid (vitamin B5), pyridoxine (vitamin B6), biotin (vitamin B7), myo-inositol (vitamin B8), folic acid (vitamin B9), cobalamin (vitamin B12), and vitamin C) and fat soluble vitamins (such as vitamin A, vitamin D, vitamin E, and vitamin K) including salts, esters or derivatives thereof. Inulin, taurine, carnitine, amino acids, enzymes, coenzymes, and the like may be useful to include in various embodiments.

In one embodiment, the composition is in the form of an oil-in-water emulsion. The emulsion may comprise substantially no protein or carbohydrate. In one embodiment, the total fat content of the oil-in-water emulsion is 5 to 40 g/100 ml, for example 5 to 30 g/100 ml, 5 to 25 g/100 ml, 10-25 g/100 ml or 10-20 g/100 ml or 15 to 25 g/100 ml. In one embodiment, the energy value of the emulsion is between 50 to 300 kcal per 100 ml, for example, 100 to 300 kcal per 100 ml, 50 to 200 kcal per 100 ml, 150 to 250 kcal per 100 ml or 160 to 200 kcal per 100 ml.

In another embodiment the composition comprising decanoic acid is delivered as part of a ketogenic diet. If the invention is delivered as part of a ketogenic diet, the ratio of total fat content:protein/carbohydrate content can be altered during therapy to achieve nutritional goals and to optimise clinical benefit. The ratio can be in the range of, for example 1:1 to 7:1, 1:1 to 5:1, for example, 1:1, 1.5:1, 2:1, 2.5:1, 3:1, 3.5:1, 4:1, 4.5:1 or 5:1.

In one embodiment the ratio is 2.25:1 to 3.9:1. In another embodiment the ratio is 2.26 to 3.8:1 or 2.7-3.4:1. In further embodiments the ratio is 3.21:1, 3.23:1, 3.24:1, 3.25:1, 3.26:1, 3.27:1, 3.28:1 or 3.29:1.

The decanoic acid or composition comprising the same may be for enteral or parenteral administration. In a preferred embodiment the composition is for oral administration.

In one embodiment the decanoic acid or composition comprising the same is in the form of a tablet, dragee, capsule, gel cap, powder, granule, solution, emulsion, suspension, coated particle, spray-dried particle or pill.

In another embodiment the decanoic acid or composition comprising the same may be in the form of a powder. The powder may, for example, be a spray-dried powder or a freeze-dried powder.

The composition may be usable for reconstitution in water.

The decanoic acid or composition comprising the same may be inserted or mixed into a food substance. The composition may be in the form of a food stuff or a feed. In one embodiment the food stuff is a human food stuff.

The decanoic acid or composition comprising the same may be in the form of a medical food. The term “medical food” as used herein refers to a food product specifically formulated for the dietary management of a medical disease or condition; for example, the medical disease or condition may have distinctive nutritional needs that cannot be met by normal diet alone. The medical food may be administered under medical supervision. The medical food may be for oral ingestion or tube feeding.

The composition comprising the decanoic acid may be in the form of a tube feed. The term “tube feed” refers to a product which is intended for introducing nutrients directly into the gastrointestinal tract of a subject by a feeding tube. A tube feed may be administered by, for example, a feeding tube placed through the nose of a subject (such as nasogastric, nasoduodenal, and nasojejunal tubes), or a feeding tube placed directly into the abdomen of a subject (such as gastrostomy, gastrojejunostomy, or jejunostomy feeding tube).

The composition comprising the decanoic acid may be in the form of a nutritional composition or a nutritional supplement. The term “nutritional supplement” refers to a product which is intended to supplement the general diet of a subject.

The composition comprising the decanoic acid may be in the form of a complete nutritional product. The term “complete nutritional product” refers to a product which is capable of being the sole source of nourishment for the subject.

In various embodiments the composition may be in the form of a beverage, mayonnaise, salad dressing, margarine, low fat spread, dairy product, cheese spread, processed cheese, dairy dessert, flavoured milk, cream, fermented milk product, cheese, butter, condensed milk product, ice cream mix, soya product, pasteurised liquid egg, bakery product, confectionary product, confectionary bar, chocolate bar, high fat bar, liquid emulsion, spray-dried powder, freeze-dried powder, UHT pudding, pasteurised pudding, gel, jelly, yoghurt, or a food with a fat-based or water-containing filling.

In yet other embodiments the composition may be used to coat a food.

The composition may in the form of a pharmaceutical composition and may comprise one or more suitable pharmaceutically acceptable carriers, diluents and/or excipients.

Examples of such suitable excipients for compositions described herein may be found in the “Handbook of Pharmaceutical Excipients, 2nd Edition, (1994), Edited by A Wade and PJ Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s) and/or solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilisers, dyes and even flavouring agents may be provided in the composition. Examples of preservatives include sodium benzoate, sorbic acid and esters of p-hydroxybenzoic acid. Antioxidants and suspending agents may be also used.

Nutritionally acceptable carriers, diluents and excipients include those suitable for human or animal consumption and that are used as standard in the food industry. Typical nutritionally acceptable carriers, diluents and excipients will be familiar to the skilled person in the art.

### Perampanel

Perampanel is a non-competitive AMPA glutamate receptor antagonist. It is marketed under the name Fycompa™ and is indicated as an adjunctive treatment of partial-onset seizures with or without secondarily generalised seizures in adult and adolescent patients with epilepsy. It is also indicated for the adjunctive treatment of primary generalised tonic-clonic seizures in adult and adolescent patients with idiopathic generalised epilepsy, and shows potential efficacy in the treatment of drug-resistant epilepsy.

The term ‘perampanel’ as used herein refers to a compound having the following structure:

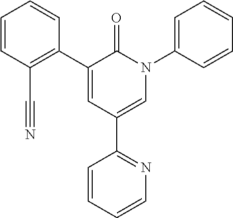 [SMILES: N#Cc1ccccc1-c1cc(-c2ccccn2)cn(-c2ccccc2)c1=O]

Perampanel has the chemical name 3-(2-Cyanophenyl)-5-(2-pyridyl)-1-phenyl-1,2-dihydropyridin-2-one. The present invention also encompasses pharmaceutically acceptable salts of perampanel.

A “pharmaceutically acceptable salt” as referred to herein, is any salt preparation that is appropriate for use in a pharmaceutical application. Pharmaceutically acceptable salts include, but are not limited to, amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane and the like; alkali metal salts, such as lithium, potassium, sodium and the like; alkali earth metal salts, such as barium, calcium, magnesium and the like; transition metal salts, such as zinc, aluminum and the like; other metal salts, such as sodium hydrogen phosphate, disodium phosphate and the like; mineral acids, such as hydrochlorides, sulfates and the like; and salts of organic acids, such as acetates, lactates, malates, tartrates, citrates, ascorbates, succinates, butyrates, valerates, fumarates and the like.

Perampanel may be administered according to individual patient response, in order to optimise the balance between efficacy and tolerability.

Preferably, perampanel is administered orally.

Perampanel at doses of 4 mg/day to 12 mg/day has been shown to be effective therapy in partial-onset seizures. Perampanel at a dose up to 8 mg/day has been shown to be effective in primary generalised tonic-clonic seizures. In one embodiment, the dosage of perampanel used in the present invention is between 4 mg/day to 12 mg/day. However, the dosage of perampanel is not limited to these dosages and may be increase or reduced depending on the response of the subject.

AMPA Receptor Inhibitor that Binds to the Same AMPA Receptor Site as Perampanel

The AMPA receptor is a non-N-methyl-D-aspartate-type (non-NMDA-type) ionotropic transmembrane receptor for glutamate that mediates fast synaptic transmission in the central nervous system, and perampanel is known to selectively inhibit AMPA receptor-mediated synaptic excitation without affecting NMDA receptor responses (Rogawski M. A., Acta Neurol Scand Suppl. 2013; (197): 19-24). Yelshanskaya, M. V., Neuron 2016, 91, 1305-1315, specifically characterizes the binding site of perampanel on the AMPA receptor and reveals that binding occurs at an allosteric site on the ion channel extracellular side.

As used herein, the expression “AMPA receptor inhibitor that binds to the same AMPA receptor site as perampanel” or “AMPA receptor inhibitor that binds to the same receptor site as perampanel” means that the AMPA receptor inhibitor binds to the same site on the AMPA receptor as that of perampanel. Perampanel and related compounds have been suggested to bind at the S1-M1 and S2-M4 linker between transmembrane and extracellular domains domains of the GluA2 subunit of the AMPA receptor. The AMPA receptor site to which perampanel binds has been characterized in Yelshanskaya, M. V., Neuron 2016, 91: 1305-1315. The specific disclosure in Yelshanskaya, M. V. of the site of the AMPA receptor to which perampanel binds is incorporated herein by reference.

A number of techniques are known in the art that are suitable for identifying and characterising agents that inhibit AMPA receptors, including determining the specific binding site of the inhibitor on the AMPA receptor. For example, electrophysiological techniques such as whole-cell patch clamp methods are suitable for assaying AMPA receptor activity and its inhibition by candidate agents in a quantitative manner. Exemplary methods for characterising AMPA receptor inhibitors, including determining the specific binding site of the inhibitors on the AMPA receptor are described in Chang et al., Brain. 2016 February; 139(2): 431-443, and Yelshanskaya, M. V. et al., Neuron 2016, 91: 1305-1315.

For determining inhibition, AMPA receptors may be expressed in suitable cells (e.g. Xenopus oocytes or HEK293 cells) and patch-clamp current recordings may be used to measure the level of inhibition of receptor current (e.g. receptor current elicited by glutamate) by a candidate agent. Quantitative determination of inhibition may be achieved by measuring the extent of current inhibition at varying concentrations of a candidate agent.

The inhibitory activity of a candidate agent may be expressed, for example, in terms of an IC₅₀ value. The IC₅₀ is the concentration of an agent that is required to give rise to a 50% reduction in the activity of the protein (e.g. a 50% reduction in AMPA receptor activity). In one embodiment the agents of the invention have an IC₅₀ value for AMPA receptor inhibition of less than 10 μM, 5 μM, 4 μM, 3 μM, 2 μM, 1 μM, 0.9 μM, 0.8 μM, 0.7 μM, 0.6 μM, 0.5 μM, 0.4 μM, 0.3 μM, 0.2 μM or 0.1 μM.

In one embodiment the AMPA receptor inhibitor that binds to the same AMPA receptor site as perampanel is a small molecule, e.g. an organic compound. The organic compound may, for example, have a molecular weight of approximately less than 900 daltons (Da). In another embodiment the AMPA receptor inhibitor is a polypeptide or protein. Preferably the AMPA receptor inhibitor that binds to the same AMPA receptor site as perampanel is a small molecule. In one embodiment the AMPA receptor inhibitor is a perampanel derivative.

### Treatment

As used herein, the term “treatment” means to administer a combination or composition as described herein to a subject having a condition in order to prevent, lessen, reduce or improve at least one symptom associated with the condition and/or to slow down, reduce or block the progression of the condition.

To “prevent” means to administer a combination or composition as described herein to a subject that is not showing any symptoms of the condition to reduce or prevent development of at least one symptom associated with the condition.

The subject to be treated may be identified as a subject that would respond to AMPA receptor inhibition. Such a subject can, for example, be identified as a subject that has previously responded to treatment with perampanel or a pharmaceutically acceptable salt thereof, or an AMPA receptor inhibitor that binds to the same AMPA receptor site as perampanel.

### Epilepsy

Epilepsy is a neurological disorder in which nerve cell activity in the brain becomes disrupted, causing seizures or periods of unusual behaviour, sensations and sometimes loss of consciousness.

AMPA receptors play a key role in the generation and spread of epileptic seizures (Rogawski et al., Acta Neurol. Scand. Suppl. 127 (197): 9-18). The receptors are present in all areas relevant to epilepsy, including the cerebral cortex, amygdala, thalamus and hippocampus. Furthermore, AMPA receptor antagonists have a broad spectrum of anticonvulsant activity in various in vitro and in vivo epilepsy models ((Rogawski, Epilepsy Curr 2011; 11: 56-63).

Owing to the ability of the combination referred to herein to optimally inhibit the AMPA receptor, the combination or composition described herein may be used to treat epilepsy.

### Amyotrophic Lateral Sclerosis

Amyotrophic lateral sclerosis (ALS), also known as Lou Gehrig's disease and motor neurone disease (MND) is the most common adult-onset motor neuron disease, and is characterised by the progressive loss of both upper and lower motor neurons resulting in muscle weakness and atrophy throughout the body. ALS may be inherited or sporadic. Typically, patients with ALS die from progressive respiratory muscle paralysis within a few years after disease onset. Excitotoxicity, a pathological process in which neurons are damaged and killed by over-activity of AMPA receptors, has been proposed to underlie ALS pathogenesis. Orally administered perampanel prevented the progression of the ALS phenotype in a mouse model of ALS (Akamatsu et al., Sci. Rep (2017) 6:28649). Owing to the ability of the combination or composition referred to herein to optimally inhibit the AMPA receptor, the compositions may be used to treat ALS.

### Ischemia

Ischemia is a restriction in blood flow to a tissue associated with a deleterious shortage in oxygen and glucose supply, e.g. hypoxia and hypoglycaemia. During ischemia, the Ca²⁺ permeability of AMPA receptors may increase, which can lead to excitotoxicity and associated neuronal cell death. Ca²⁺-permeable AMPA receptors have been shown to be highly expressed in CA1 pyramidal neurons—a region of the hippocampus that is more vulnerable to cell death following an ischemic event than other hippocampal regions. AMPA receptor antagonists, such as NBQX, have been demonstrated to be beneficial in preventing neuronal loss in animal models of ischemia (Chang et al., (2012) European Journal of Neuroscience, 35, 1908-1916). Owing to the ability of the combination or composition referred to herein to optimally inhibit the AMPA receptor, the combination or composition described herein may be used to treat ischemia.

### Cancer

A link between the MCT ketogenic diet, AMPA receptors and cancer treatment has been established by studies demonstrating that human glioblastoma cells express increased levels of AMPA receptors (Choi, J., et al., Glioblastoma cells induce differential glutamatergic gene expressions in human tumor-associated microglia/macrophages and monocyte-derived macrophages. Cancer Biol Ther, 2015. 16(8): p. 1205-13), and inhibition of AMPA receptors suppresses migration and proliferation of glioblastoma multiforme cells (GBM) (Ishiuchi, S., et al., Ca2+-permeable AMPA receptors regulate growth of human glioblastoma via Akt activation. J Neurosci, 2007. 27(30): p. 7987-8000, Ishiuchi, S., et al., Blockage of Ca(2+)-permeable AMPA receptors suppresses migration and induces apoptosis in human glioblastoma cells. Nat Med, 2002. 8(9): p. 971-8, Yoshida, Y., et al., Serum-dependence of AMPA receptor-mediated proliferation in glioma cells. Pathol Int, 2006. 56(5): p. 262-71.) and other cancer cells (von Roemeling, C. A., et al., Neuronal pentraxin 2 supports clear cell renal cell carcinoma by activating the AMPA-selective glutamate receptor-4. Cancer Res, 2014. 74(17): p. 4796-810). Furthermore, perampanel has been shown to be a potentially chemotherapeutically active adjuvant in a single case study of GBM treatment (Rosche, J., et al., [Perampanel in the treatment of a patient with glioblastoma multiforme without IDH1 mutation and without MGMT promotor methylation]. Fortschr Neurol Psychiatr, 2015. 83(5): p. 286-9). These studies thus suggest that AMPA receptor inhibition through a combination of decanoic acid and perampanel has potential to provide an adjunctive cancer treatment.

### Alzheimer's Disease

There is strong evidence that amyloid β(Aβ) increases AMPA receptor currents and triggers subunit internalization, a theory that directly links glutamate receptor hyperactivity to neurotoxicity and memory loss in Alzheimer's disease. Aβ has been shown to interact with β adrenergic receptors which regulate gene expression and the activity of various receptors including AMPA-type glutamate receptors via the cAMP/PKA signaling cascade (Wang, D., et al., Binding of amyloid beta peptide to beta2 adrenergic receptor induces PKA-dependent AMPA receptor hyperactivity. FASEB J, 2010. 24(9): p. 3511-21, Wisely, E. V., Y. K. Xiang, and S. Oddo, Genetic suppression of beta2-adrenergic receptors ameliorates tau pathology in a mouse model of tauopathies. Hum Mol Genet, 2014. 23(15): p. 4024-34). Phosphorylation of AMPA receptor GluA1 subunits by PKA has been shown to increase channel opening probability which results in augmented calcium entry into the cell (Banke, T. G., et al., Control of GluR1 AMPA receptor function by cAMP-dependent protein kinase. J Neurosci, 2000. 20(1): p. 89-102). Indeed, numerous studies have shown that the addition of Aβ to neuronal cultures causes neurotoxicity by strengthening calcium-dependent AMPA-receptor generated currents (Whitcomb, D. J., et al., Intracellular oligomeric amyloid-beta rapidly regulates GluA1 subunit of AMPA receptor in the hippocampus. Sci Rep, 2015. 5: p. 10934). This suggests that Aβ induced excitotoxicity could contribute to the widespread neuronal death in Alzheimer's disease. In addition to ketones providing energy to glucose resistant neurons, the MCT ketogenic diet might therefore improve neuronal survival through the inhibition of AMPA receptors by decanoic acid. In addition, there is evidence that Aβ treatment triggers the internalization of GluA2 subunits, the only AMPA receptor subunit type that confers calcium impermeability. Internalization of GluA2 could therefore further increase total calcium influx at the post-synapse which could further increase inflammation and neurotoxicity (Beppu, K., et al., Expression, subunit composition, and function of AMPA-type glutamate receptors are changed in activated microglia; possible contribution of GluA2 (GluR-B)-deficiency under pathological conditions. Glia, 2013. 61(6): p. 881-91, Noda, M., Dysfunction of Glutamate Receptors in Microglia May Cause Neurodegeneration. Curr Alzheimer Res, 2016. 13(4): p. 381-6) suggesting a role for AMPA receptor antagonists in the treatment of Alzheimer's disease. Owing to the ability of the combination or composition referred to herein to optimally inhibit the AMPA receptor, the combination or composition referred to herein may be used to treat Alzheimer's disease.

### Administration

The combination, product or composition described herein may be administered enterally or parenterally.

Preferably, the product, combination or composition is administered enterally.

Enteral administration may be oral, gastric, and/or rectal.

In general terms, administration of the combination or composition described herein may, for example, be by an oral route or another route into the gastro-intestinal tract, for example the administration may be by tube feeding.

The subject may be a mammal such as a human, canine, feline, equine, caprine, bovine, ovine, porcine, cervine and primates. Preferably the subject is a human.

### EXAMPLES

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

### Example 1—Methods

### Direct AMPA Receptor Current Recording.

As previously described (Chang et al. 2016. Brain 139: 431-43), AMPA receptor subunits (GluA2/3, GluA1/2, and GluA3) were expressed in Xenopus oocytes and agonist elicited inward currents were used to measure inhibition by decanoic acid (Sigma Ltd) and perampanel (Apexmol Technology Co. Ltd).

### Rat Hippocampal PTZ Seizure-Like Activity Analysis

Seizure-like activity was induced, as previously described (Chang et al. 2016. Brain 139: 431-43), in rat entorhinal cortex-hippocamal slices by application of PTZ (2 mM) to the perfusate and [K⁺] was increased (to 6 mM). Perampanel (100 or 500 nM) or DMSO was applied to the perfusate, and then decanoic acid (Sigma Ltd) was applied at increasing concentrations at 10 minutes intervals. The change in the frequency of the discharges was measured at minute intervals, averaged every 5 minutes, and normalised to baseline. Data were analyzed using Origin (OriginLab Corporation, MA USA) and SPSS (IBM, UK) with IC₅₀ values calculated from fitting data values with a Hill plot. Statistical analysis was performed using a one-way ANOVA with two-way Dunnett's post hoc test.

### Example 2—Direct Inhibition of AMPA Receptors by Perampanel and Decanoic Acid

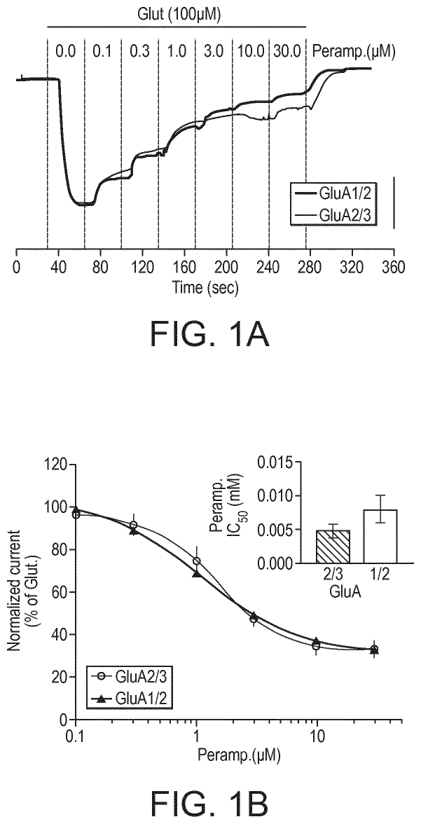

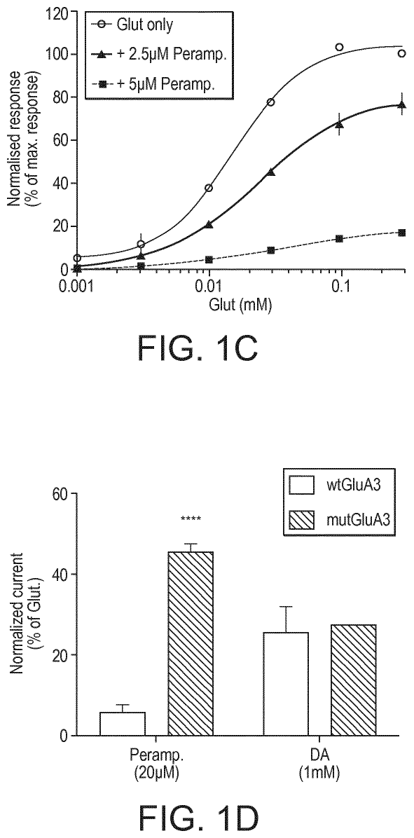

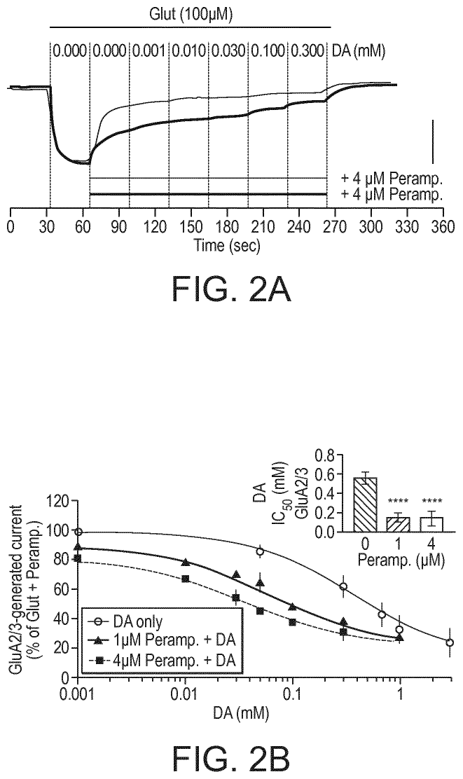

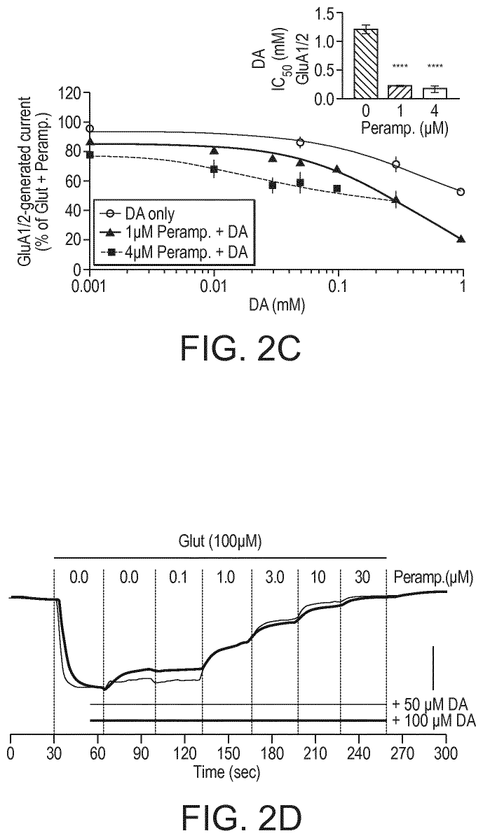

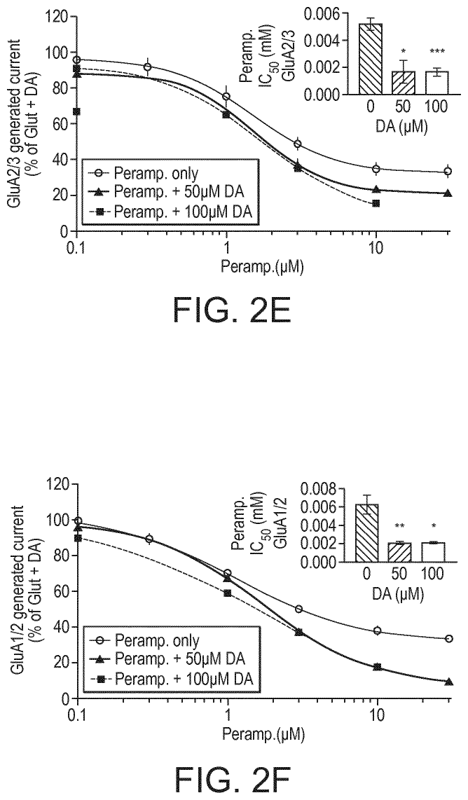

We first determined the effect of perampanel on currents elicited by glutamate (100 μM) application to Xenopus laevis oocytes expressing GluA1/2 or 2/3, the two most common AMPA receptor subunit combinations found in the hippocampus (Jacob and Weinberg, 2015. Hippocampus 25: 798-812). Perampanel inhibited AMPA receptor currents with an IC₅₀ value of 1.51 μM against GluA2/3 (CI 0.96 to 2.37) and 1.12 μM against GluA1/2 (CI 0.79 to 1.58) (FIG. 1A,B). In addition, perampanel inhibited AMPA receptor currents in a non-competitive manner, with increasing glutamate concentrations not reversing perampanel inhibition (FIG. 1C). Perampanel also reduced glutamate evoked maximal responses to 75.2% (at 2.5 μM) and 16.9% (at 5 μM). These data directly show, for the first time, the inhibition of AMPA receptors by perampanel through a non-competitive inhibition without subunit specificity.

Recent studies have suggested distinct binding sites for perampanel and decanoic acid on the AMPA receptor (Chang P, Augustin K, Boddum K, Williams S, Sun M, et al. 2016. Brain 139: 431-43; Yelshanskaya M V, Singh A K, Sampson J M, Narangoda C, Kurnikova M, Sobolevsky A I. 2016. Neuron 91: 1305-15). Perampanel and related compounds have been suggested to bind at the S1-M1 and S2-M4 linker between transmembrane and extracellular domains (Yelshanskaya M V, Singh A K, Sampson J M, Narangoda C, Kurnikova M, Sobolevsky A I. 2016. Neuron 91: 1305-15). In contrast, decanoic acid modelling suggests binding to the M3 region (Chang P, Augustin K, Boddum K, Williams S, Sun M, et al. 2016. Brain 139: 431-43). To confirm this, we expressed a GYKI resistant subunit GluA3 mutant (11) and assessed it for sensitivity to perampanel and decanoic acid (FIG. 1C). Perampanel (20 μM) reduced the wild type AMPA receptor (GluA3) glutamate-induced currents by 94.7%, but the mutant receptor by only 54.3% (p<0.0001). In contrast, decanoic acid (1 mM) reduced wild type and mutant glutamate-induced currents by 74.5% (SEM 6.4) and 72.8% (SEM 0.3) respectively, strongly supporting that decanoic acid interacts with AMPA receptors at a different site.

To investigate the possibility of synergism between decanoic acid and perampanel, we tested AMPA receptor (GluA2/3) sensitivity to decanoic acid within the concentration range observed in patients receiving the MCT ketogenic diet (12), at two concentrations of perampanel (1 μM and 4 μM) (FIG. 1E,F). In these experiments, decanoic acid potency was increased by perampanel, lowering IC₅₀ for GluA2/3 from 0.52 mM (in the absence of perampanel) to 0.10 mM or 0.04 mM in the presence of 1 μM or 4 μM perampanel respectively (p<0.0001). This effect was also observed in GluA1/2 AMPA receptors (FIG. 1G), the decanoic acid IC₅₀ for GluA1/2 was reduced from 0.92 mM (in the absence of perampanel) to 0.21 mM and 0.09 mM in the presence of 1 μM or 4 μM respectively (p<0.0001). Repeating this approach using perampanel with two concentrations of decanoic acid (50 mM and 100 mM) (FIG. 1H, I, J), also showed a significant increase in the potency of perampanel, decreasing IC₅₀ values from 5.1 μM (in the absence of DA) to 1.7 μM and 1.6 μM in the presence of DA at 50 mM and 100 mM respectively in GluA2/3 and from 6.2 μM in GluA1/2 to 2.1 μM and 2.2 μM, respectively (both p<0.0001). These results imply synergistic inhibition of AMPA receptors by decanoic acid and perampanel.

### Example 3—Effects of Perampanel and Decanoic Acid on Seizure Models

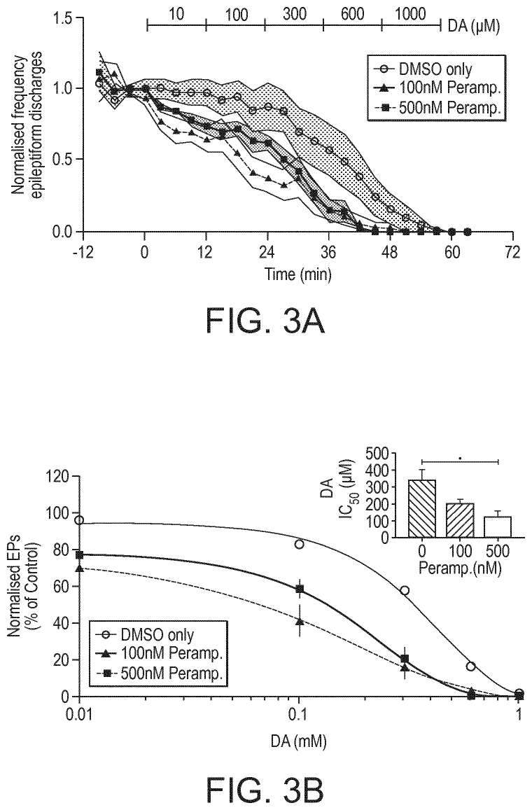

We then investigated the synergistic effect of perampanel and decanoic acid on seizures in rat hippocampal slices, with seizure-like activity generated through treatment with pentelenetetrazol (PTZ). In these experiments, increasing concentrations of decanoic acid reduced epileptiform activity at 300 μM with a block at 1000 μM (FIG. 3A), consistent with earlier data (Chang P, Augustin K, Boddum K, Williams S, Sun M, et al. 2016. Brain 139: 431-43). Repeating the assay with added perampanel (100 and 500 nM) with data normalised to perampanel alone, showed enhanced epileptiform control from 10-1000 μM decanoic acid. Modelling these data shows that decanoic acid (10 μM) combined with perampanel (100 nM) caused a significant reduction in seizure activity from 95.6% (CI 80.8 to 110.4) in the absence of decanoic acid to 76.8% (CI 63.1 to 90.4, p=0.048) in its presence, and a block in activity at 600 μM. A similar activity is also shown at higher perampanel (500 nM) concentrations, reducing baseline (10 μM) inhibition to 69.6% (CI 53.1 to 86, p=0.015). These data also demonstrate a reduced IC₅₀ for decanoic acid from 352 μM (CI: 200.1 to 621.5) to 196 μM (145.2 to 264.8) and 122 μM (49.13 to 302.9, p=0.0252) at 100 nM and 500 nM respectively.

### SUMMARY

Data presented here investigates the use of a combination comprising decanoic acid and perampanel for the treatment of epilepsy. Data provided here illustrate a significant ˜3-fold reduction in the IC₅₀ value for perampanel against the two most common AMPA receptor subunit combinations (GluA2/3 and GluA1/2) demonstrating a direct synergistic inhibition of these receptors at a molecular level. We show that the synergistic effects of combinatory treatment is also evident in an ex vivo seizure model, where epileptiform activity is induced by application of PTZ.

Potential synergistic effects between perampanel and decanoic acid depend on suitable levels of each compound. In early studies on the levels of medium chain fatty acids in the peripheral blood of patients on the MCT ketogenic diet, the concentration of decanoic acid blood is around 87 to 552 μM with an average of 157 μM (Haidukewych D, Forsythe W I, Sills M. 1982. Clin Chem 28: 642-5; Sills M A, Forsythe W I, Haidukewych D. 1986. Arch Dis Child 61: 1173-7).

In rodent models, the ratio of decanoic acid in blood plasma to brain in animal models is around 0.7 (Wlaz P, Socala K, Nieoczym D, Zarnowski T, Zarnowska I, et al. 2015. Prog Neuropsychopharmacol Biol Psychiatry 57: 110-6). Extrapolating this ratio to human brain suggests that decanoic acid is likely to be present in the brain at average concentrations of around 110 μM. Our data suggests that 100 μM decanoic acid results in a 3-fold increase in the inhibition of the AMPA receptor by perampanel which translates to an even greater impact on seizure activity.

These data support the potential clinical relevance of this synergistic effect in patients. This may provide a means for reducing the side effect profile of perampanel or cause a significant improvement in treating diseases in which inhibition of AMPA receptors is beneficial, such as the treatment of seizure control. The synergistic inhibition of AMPA receptors through combinatory perampanel and decanoic acid may therefore provide both improved treatment and reduced side effects in patient populations. Given the concerning side effects of perampanel, particularly the cognitive and psychiatric side effects observed at higher doses reported by Rugg-Gunn, F. in Epilepsia. 2014 January; 55 Suppl 1:13-5, this is a significant advantage of the present invention. The inventors believe that the combination of decanoic acid and perampanel (or a pharmaceutically acceptable salt thereof) may allow the use of lower doses of perampanel in the treatment of epilepsy and thereby reduce severity and/or prevalence of its side-effects.

1. A method for treating epilepsy comprising administering a composition comprising decanoic acid in combination with perampanel, or a pharmaceutically acceptable salt thereof, or an AMPA receptor inhibitor that binds to the same AMPA receptor site as perampanel to an individual in need of same.

2. A method for treating epilepsy comprising administering perampanel or a pharmaceutically acceptable salt thereof combination with decanoic acid to an individual in need of same.

3. A method for use in treating epilepsy comprising administering an AMPA receptor inhibitor that binds to the same AMPA receptor site as perampanel in combination with decanoic acid to an individual in need of same.

4. Method according to claim 1, wherein the decanoic acid, and the perampanel, or a pharmaceutically acceptable salt thereof, are administered separately or sequentially, or wherein the decanoic acid and the AMPA receptor inhibitor are administered separately or sequentially.

5-9. (canceled)

10. Method according to claim 4, wherein the use is for treating a subject that has been identified as a subject that would respond to AMPA receptor inhibition.

11. Method according to claim 1, wherein the decanoic acid is in the form of a triglyceride.

12. Method according to claim 1, wherein the decanoic acid is comprised in an oil-in-water emulsion, a powder or a food stuff.

13. Method according to claim 1, wherein the decanoic acid is comprised within a medical food, a tube feed, a nutritional composition or a nutritional supplement.

14. Method according to claim 1, wherein the composition further comprises one or more pharmaceutically acceptable carrier, diluents and/or excipients.

15. (canceled)
